(19) United States
(12) Patent Application Publication
(10) Pub. No.: US 2026/0159798 A1
(43) Pub. Date: Jun. 11, 2026

(54) ROTARY BIOLOGICAL CULTURE AND OBSERVATION APPARATUS

(71) Applicant: Shandong University, Jinan City, SD (US)

(72) Inventors: Keliang WU, Jinan City (CN); Cheng Li, Jinan City (CN)

(21) Appl. No.: 19/241,481

(22) Filed: Jun. 18, 2025

(30) Foreign Application Priority Data
Dec. 11, 2024 (CN) 202411816028.2

(51) Int. Cl. C12M 3/04; C12M 1/00; C12M 1/12; C12M 1/34

(52) U.S. Cl. CPC C12M 27/10; C12M 23/34; C12M 23/38; C12M 29/20; C12M 29/24; C12M 37/04; C12M 41/06; C12M 41/12; C12M 41/34; C12M 41/36

(57) ABSTRACT

A rotary biological culture and observation apparatus is provided. The rotary biological culture and observation apparatus includes a control-display terminal and a control host, and further includes an environmental regulation system, and culture dish rotation systems and an imaging system both arranged in the environmental regulation system. The environmental regulation system includes a culture chamber, the culture chamber includes a chamber body and a door connected to the chamber body, and multiple culture dish rotation dish systems are distributed in the chamber body circumferentially.

### CROSS-REFERENCE TO RELATED APPLICATION

This patent application claims the benefit and priority of Chinese Patent Application No. 202411816028.2, filed with the China National Intellectual Property Administration on Dec. 11, 2024, the disclosure of which is incorporated by reference herein in its entirety as part of the present application.

### TECHNICAL FIELD

The present disclosure relates to the technical field of biological culture apparatuses, and in particular to a rotary biological culture and observation apparatus.

### BACKGROUND

In the field of biological cell culture, the culture of biological samples is usually performed by a static culture method (biological culture samples suspend statically in nutrients or adsorbed on inner walls of culture vessels), and developmental states of the biological samples are monitored and evaluated by means of microscope observation. The static culture method may result in the developmental retardation or death of cells of the biological samples due to insufficient nutrient exchange between the cells and a culture solution and culture environments that do not meet the conditions of original organisms when the cells develop into cell aggregates. When observing the developmental states of the biological samples by a microscope in a non-culture environment, the cells of the biological samples may be subjected to developmental retardation or death due to sudden changes in a developmental temperature environment and a gas concentration environment. Therefore, in order to avoid the above problems, the conventional technology adopts the combination of a rotary biological culture method and the microscope, utilizes centrifugal forces generated by rotation and hydrodynamic properties to optimize cell growth environments, and can support the development of cells of the biological samples into cell aggregates. Moreover, by integrating microscope imaging, real-time dynamic image monitoring of developmental processes of the biological samples can be achieved without damaging the development environments.

### SUMMARY

An objective of the present disclosure is to provide a rotary biological culture and observation apparatus to solve the above technical problems.

In order to achieve the above objective, the present disclosure provides a rotary biological culture and observation apparatus, including a control-display terminal and a control host, the control-display terminal being electrically connected to the control host, the rotary biological culture and observation apparatus further including an environmental regulation system, culture dish rotation systems and an imaging system, the culture dish rotation systems and the imaging system both being arranged inside the environmental regulation system, where the environmental regulation system includes a culture chamber, the culture chamber includes a chamber body and a door connected to the chamber body, and multiple culture dish rotation systems are distributed in the chamber body circumferentially.

In some embodiments, a sealing gasket is arranged at a contact position between the chamber body and the door, and the culture chamber is provided with a vent.

In some embodiments, a temperature regulation subsystem is arranged inside the culture chamber, the temperature regulation subsystem includes a door temperature controller and a chamber body temperature controller, the door temperature controller is electrically connected to a first heating plate secured within the door, the chamber body temperature controller is electrically connected to a second heating plate secured within the chamber body, and the door temperature controller and the chamber body temperature controller are both electrically connected to the control host.

In some embodiments, a gas concentration regulation subsystem is arranged inside the culture chamber, the gas concentration regulation subsystem includes an intake controller, a detection assembly, and a homogenizing fan, the intake controller, the detection assembly and the homogenizing fan are all electrically connected to the control host, the homogenizing fan and the detection assembly are both mounted inside the chamber body, the detection assembly includes an oxygen sensor and a carbon dioxide sensor, and the intake controller includes a nitrogen gas port, an oxygen gas port, a carbon dioxide gas port, and a mixed gas outlet;

oxygen concentration regulation operations are as follows:
the nitrogen gas port is configured to be opened while the oxygen gas port is closed, and nitrogen gas is continuously fed to discharge oxygen gas from the chamber body to reduce a concentration of the oxygen gas in the chamber body; and the oxygen gas port is configured to be opened while the nitrogen gas port is closed, and the oxygen gas is continuously fed to increase the concentration of the oxygen gas in the chamber body;

carbon dioxide regulation operations are as follows:
the carbon dioxide gas port is configured to be closed while the nitrogen gas port is opened, and the nitrogen gas is continuously fed into the chamber body to reduce a concentration of carbon dioxide in the chamber body; and
the carbon dioxide gas port is configured to be opened, and the carbon dioxide is continuously fed into the chamber body to increase the concentration of the carbon dioxide in the chamber body.

In some embodiments, each of the multiple culture dish rotation systems includes a dish holder to be connected to a culture dish, and a protective housing, the dish holder is mounted at an end of a hollow shaft, the hollow shaft is connected to the protective housing by means of two bearings, the hollow shaft between the two bearings is provided with a rotation driven gear, the rotation driven gear meshes with a rotation driving gear, the rotation driving gear is connected to a transmission shaft, the transmission shaft passes through the protective housing to be connected to a rotation drive motor, and the rotation drive motor is mounted on the protective housing;

a photoelectric switch is mounted on a rear cover plate of the protective housing, a detection plate is mounted on the hollow shaft, and the detection plate is arranged opposite to the photoelectric switch; and

the rotation drive motor and the photoelectric switch are both electrically connected to the control host.

In some embodiments, the imaging system includes light source assemblies and microscope assemblies, the light source assemblies are arranged opposite to the microscope assemblies.

In some embodiments, each of the light source assemblies includes an adjusting plate and a light source, the light source is mounted on the door and arranged opposite to the hollow shaft, the door is provided with multiple elongated adjusting through-holes distributed circumferentially, a limit post on the adjusting plate is arranged in a corresponding one of the multiple elongated adjusting through-holes, and the adjusting plate is arranged between the light source and the culture dish; and

the light source is electrically connected to the control host.

In some embodiments, each of the microscope assemblies includes an image grabber, a position adjusting mechanism, and an imaging adjusting mechanism, the position adjusting mechanism includes a rotating platform mounted in a middle part of the chamber body, a telescopic motor is mounted on the rotating platform, the imaging adjusting mechanism is mounted at a telescopic end of the telescopic motor and includes a microscope and a focusing driven gear and a magnification adjusting driven gear both arranged on the microscope, the focusing driven gear meshes with a focusing driving gear, the focusing driving gear is arranged on a focusing motor, the magnification adjusting driven gear meshes with a magnification adjusting driving gear, the magnification adjusting driving gear is arranged on a magnification adjusting motor, the magnification adjusting motor and the focusing motor are both mounted on a mounting bracket, the mounting bracket is arranged at the telescopic end of the telescopic motor, the image grabber is mounted on the mounting bracket, and the image grabber is arranged opposite to the microscope; and

the image grabber, the rotating platform, the telescopic motor, the magnification adjusting motor and the focusing motor are all electrically connected to the control host.

Therefore, by using the rotary biological culture and observation apparatus, embodiments of the present disclosure achieve the following beneficial effects.

(1) The rotation of culture dishes is achieved by means of the culture dish rotation systems. Shear forces of fluids on cells can be reduced significantly by setting corresponding rotational speeds, thereby protecting the cells from damage. Cultured cells are kept in centers of the culture dishes during rotation, which simulates a microgravity environment in space, helping the cells to grow without restriction in a three-dimensional environment that is similar to the in vivo environment, and facilitating cell differentiation and tissue formation. During rotation, nutrients and oxygen in a culture medium can be evenly distributed around the cells to ensure consistency of nutrient supply in cell populations and promote uniform growth of the cells. Gas is directly dissolved in the culture solution through rotation, without the need for a bubble form, so that oxygen transfer efficiency is increased while cellular damage caused by bubbles is avoided.

(2) Microscopic imaging is achieved while the culture environments are maintained, i.e., images of cultured cells are acquired during rotation to avoid irreversible damage to cells caused by changes in the culture environments. By high-frequency image capture, real-time developmental dynamic image monitoring is achieved without missing key details of development.

The technical solution of the present disclosure will be further described in detail below with reference to the accompanying drawings and embodiments.

### BRIEF DESCRIPTION OF THE DRAWINGS

FIG. 1 is a schematic diagram of an external structure of a rotary biological culture and observation apparatus according to the present disclosure;

FIG. 2 is a schematic diagram of an internal structure of the rotary biological culture and observation apparatus according to the present disclosure;

FIG. 3 is a schematic structural diagram of a culture dish rotation system according to the present disclosure;

FIG. 4 is a cross-sectional view of the culture dish rotation system according to the present disclosure;

FIG. 5 is a schematic diagram of a mounting position of a photoelectric switch according to the present disclosure;

FIG. 6 is a principle diagram of a concentration regulation subsystem according to the present disclosure;

FIG. 7 is a principle diagram of a temperature regulation subsystem according to the present disclosure; and

FIG. 8 is a schematic structural diagram of a microscope assembly according to the present disclosure.

Reference signs: 1 environmental regulation system; 11 temperature regulation subsystem; 111 door temperature controller; 112 chamber body temperature controller; 113 first heating plate; 114 second heating plate; 12 concentration regulation subsystem; 121 intake controller; 121a oxygen gas port; 121b carbon dioxide gas port; 121c nitrogen gas port; 121d mixed gas outlet; 122 detection assembly; 123 homogenizing fan; 13 culture dish rotation system; 131 culture dish; 132 dish holder; 133 hollow shaft; 134 protective housing; 135 rotation driven gear; 136 rotation driving gear; 137 transmission shaft; 138 rotation drive motor; 139 detection plate; 1310 photoelectric switch; 1311 bearing; 14 culture chamber; 141 chamber body; 142 door; 143 sealing gasket; 144 vent; 2 imaging system; 21 light source assembly; 211 adjusting plate; 212 light source; 213 elongated adjusting through-hole; 214 limit post; 22 microscope assembly; 221 microscope; 222a focusing motor; 222b magnification adjusting motor; 223 focusing driven gear; 224 magnification adjusting driven gear; 225 image grabber; 226 telescopic motor; 227 rotating platform; 3 control host; 4 control-display terminal.

### DETAILED DESCRIPTION OF THE EMBODIMENTS

### EXAMPLES

It should be noted that, in the description of the present disclosure, orientation or position relationships indicated by terms such as “upper”, “lower”, “inner”, and “outer” are based on orientation or position relationships shown in the drawings, or are orientation or position relationships to which the inventive product is customarily placed when in use, which are merely used for convenience of describing the present disclosure and simplifying the description, rather than indicating or implying that an indicated apparatus or element needs to have a specific orientation or be constructed and operated in a specific orientation, and therefore cannot be understood as a limitation on the present disclosure. In the description of the present disclosure, it should be further noted that unless otherwise explicitly specified and defined, the terms “arrange”, “mount” and “connect” should be understood in a broad sense, for example, they may be a fixed connection, a detachable connection, or an integrated connection, may be a mechanical connection or an electrical connection, may be a direct connection, an indirect connection by means of an intermediary, or may be an internal communication between two elements. For those of ordinary skill in the art, the specific meanings of the terms mentioned above in the disclosure should be construed according to specific circumstances.

Embodiments of the present disclosure will be described in detail below with reference to the accompanying drawings.

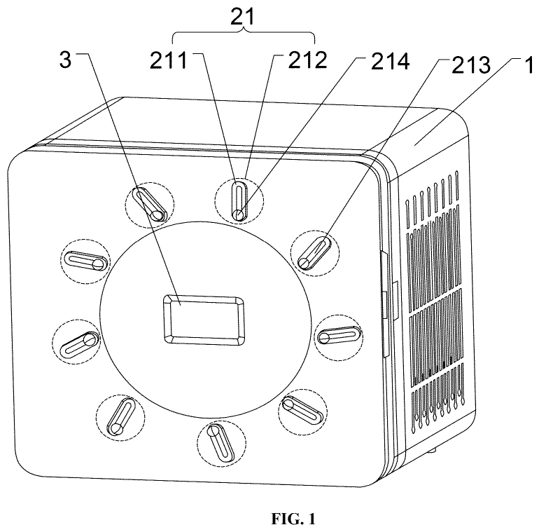

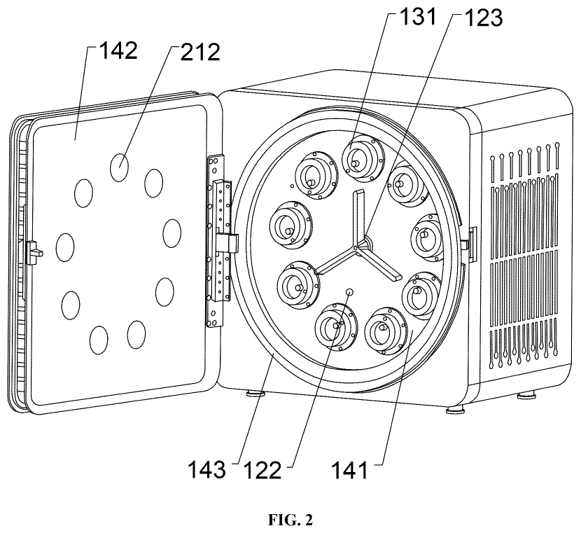

As shown in FIGS. 1 and 2, a rotary biological culture and observation apparatus includes a control-display terminal 4, a control host 3, an environmental regulation system 1, culture dish rotation systems 13 and an imaging system 2. The culture dish rotation systems 13 and the imaging system 2 are both arranged in the environmental regulation system 1. The control-display terminal 4 is electrically connected to the control host 3. In this embodiment, the control-display terminal 4 is a computer, which is an existing device. The computer can display images captured by an image grabber 225 in a microscope assembly. The computer can control the environmental regulation system 1, the culture dish rotation systems 13 and the imaging system 2 to work by means of the control host 3.

The environmental regulation system 1 includes a culture chamber 14, a temperature regulation subsystem 11, and a gas concentration regulation subsystem 12. The culture chamber 14 includes a chamber body 141 and a door 142 connected to the chamber body 141. A sealing gasket 143 is arranged at a contact position between the chamber body 141 and the door 142. The sealing gasket 143 ensures gas tightness of the culture chamber 14 when the door 142 is closed, and the culture chamber 14 is provided with a vent 144 for discharging excess gas in the process of regulating a concentration of gas in the chamber body 141.

The temperature regulation subsystem 11 is arranged inside the culture chamber 14 and configured for providing a stable temperature development environment for biological culture. As shown in FIG. 7, the temperature regulation subsystem 11 includes a door temperature controller 111 and a chamber body temperature controller 112. The door temperature controller 111 is electrically connected to a first heating plate 113 secured within the door 142, the chamber body temperature controller 112 is electrically connected to a second heating plate 114 secured within the chamber body 141, and the door temperature controller 111 and the chamber body temperature controller 112 are both electrically connected to the control host 3.

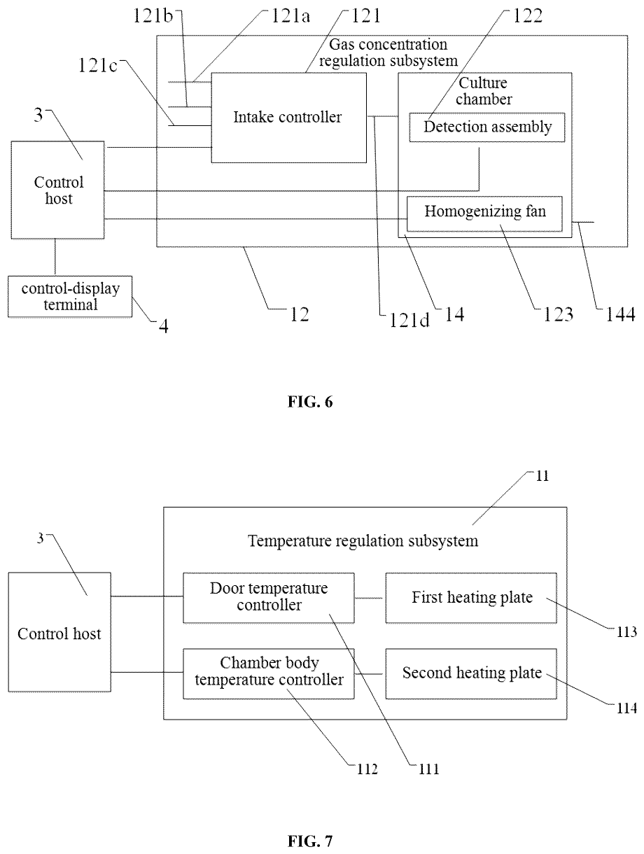

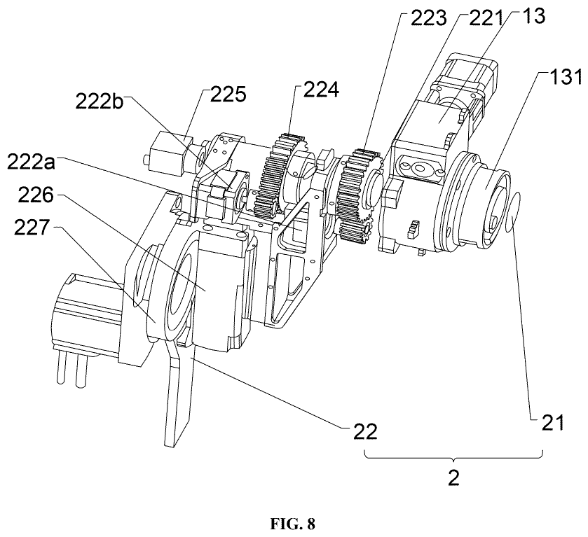

The gas concentration regulation subsystem 12 is arranged inside the culture chamber 14 and configured for providing a stable gas concentration development environment for biological culture. As shown in FIG. 6, the gas concentration regulation subsystem 12 includes an intake controller 121, a detection assembly 122, and a homogenizing fan 123. The intake controller 121, the detection assembly 122, and the homogenizing fan 123 are all electrically connected to the control host 3. The homogenizing fan 123 and the detection assembly 122 are both mounted inside the chamber body 141 and configured for mixing gases in the culture chamber 14 and detecting concentrations of the corresponding gases, respectively. The detection assembly 122 includes an oxygen sensor and a carbon dioxide sensor, where other gas sensors may be added according to the actual situations, thus providing a development environment with stable oxygen concentration and carbon dioxide concentration. The corresponding gases are mixed according to the set concentration parameters to obtain a gas mixture. The detection assembly 122 detects gas concentrations of the corresponding gases in the culture chamber 14. The detected gas concentrations are compared with the set gas concentration parameters and fed back to the control host 3. The control host 3 regulates the intake controller 121 accordingly to replenish a corresponding gas into the culture chamber 14. The excess gas is discharged from the vent 144 of the culture chamber 14 until the concentration of the corresponding gas reaches a set concentration. The intake controller 121 includes a nitrogen gas port 121c, an oxygen gas port 121a, a carbon dioxide gas port 121b, and a mixed gas outlet 121d, and the mixed gas outlet 121d is connected to the chamber body 141.

Oxygen concentration regulation operations are as follows:
the nitrogen gas port 121c is opened while the oxygen gas port 121a is closed, and nitrogen gas is continuously fed to discharge oxygen gas from the chamber body 141 to reduce a concentration of the oxygen gas in the chamber body 141; and the oxygen gas port 121a is opened while the nitrogen gas port 121c is closed, and oxygen gas is continuously fed to increase the concentration of the oxygen gas in the chamber body 141.

Carbon dioxide regulation operations are as follows:
the carbon dioxide gas port 121b is closed while the nitrogen gas port 121c is opened, and nitrogen gas is continuously fed into the chamber body 141 to reduce a concentration of carbon dioxide in the chamber body 141; and
the carbon dioxide gas port 121b is opened, and carbon dioxide is continuously fed into the chamber body 141 to increase the concentration of the carbon dioxide in the chamber body 141.

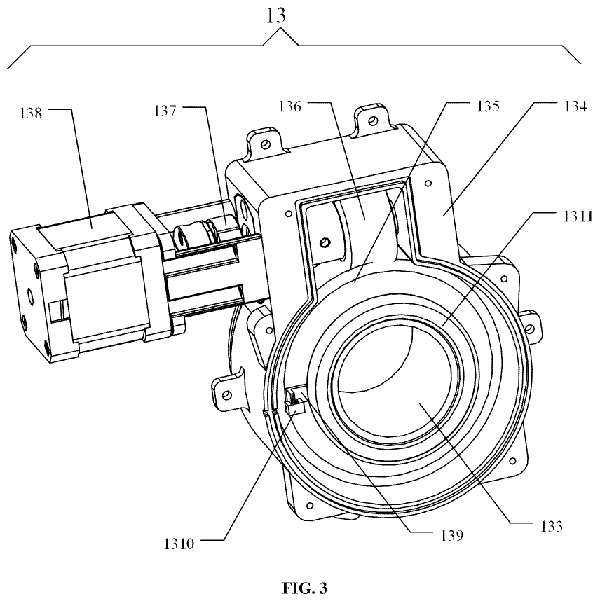

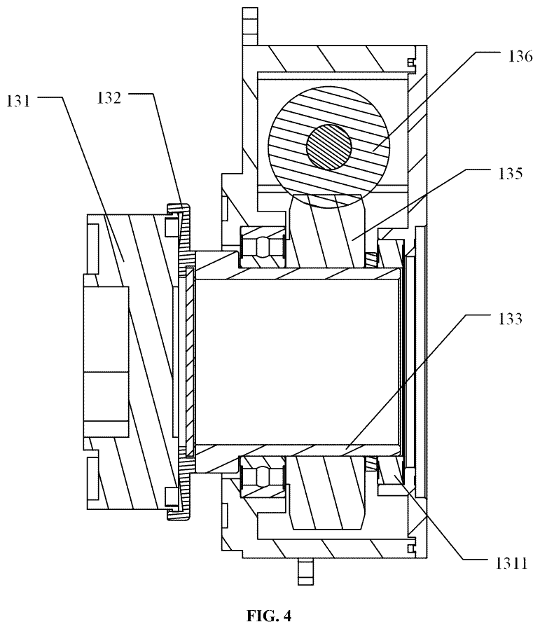

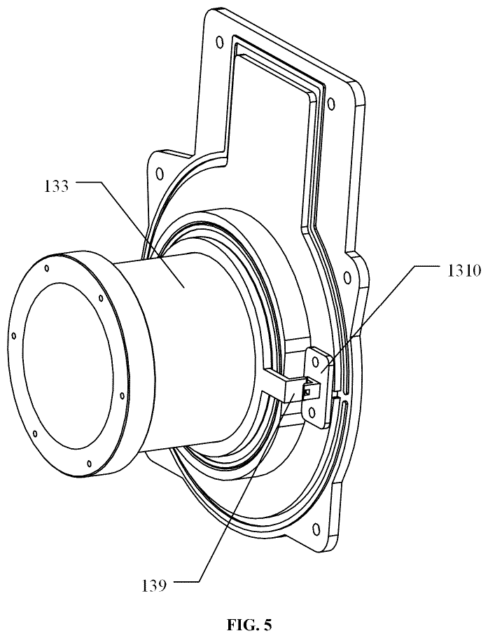

Multiple culture dish rotation systems 13 are circumferentially distributed inside the chamber body 141 to provide rotary culture environments for the development of biological samples, and centrifugal forces generated by rotation and hydrodynamic properties enable an increase in the biological cell nutrient exchange rate. As shown in FIGS. 3 and 4, the culture dish rotation system 13 includes a dish holder 132 to be connected to a culture dish 131, and a protective housing 134. The culture dish 131 contains a culture and a culture solution. The culture dish 131 is provided with a gas permeable film, which can ensure gas exchange between the inside and the outside of the culture dish while preventing liquid spillage. The dish holder 132 is mounted at an end of a hollow shaft 133, and light passing through the culture dish 131 can propagate through the hollow shaft 133. The hollow shaft 133 is connected to the protective housing 134 by means of two bearings 1311, the hollow shaft 133 between the two bearings is provided with a rotation driven gear 135, the rotation driven gear 135 meshes with a rotation driving gear 136, the rotation driving gear 136 is connected to a transmission shaft 137, the transmission shaft 137 passes through the protective housing 134 to be connected to a rotation drive motor 138, and the rotation drive motor 138 is mounted on the protective housing 134. A photoelectric switch 1310 is mounted on a rear cover plate of the protective housing 134, a detection plate 139 is mounted on the hollow shaft 133, and the detection plate 139 is arranged opposite to the photoelectric switch 1310. The rotation drive motor 138 and the photoelectric switch 1310 are both electrically connected to the control host 3. The photoelectric switch 1310 detects rotational speed of the detection plate 139 in real time and feeds it back to the control host 3, the control host 3 corrects rotation errors of the culture dish 131 in real time to achieve accurate control of the rotational speed.

The imaging system 2 includes a light source assembly 21 and a microscope assembly 22, the light source assembly 21 is arranged opposite to the microscope assembly 22. The light source assembly 21 provides light for imaging of a biological sample under a microscope 221, and includes an adjusting plate 211 and a light source 212.The light source 212 is mounted on the door 142 and arranged opposite to the hollow shaft. The door 142 is provided with multiple elongated adjusting through-holes 213 distributed circumferentially, a limit post 214 on the adjusting plate 211 is arranged in a corresponding elongated adjusting through-hole 213, the adjusting plate 211 is arranged between the light source 212 and the culture dish 131, and the light source 212 is electrically connected to the control host 3. The adjusting plate 211 can adjust the light source 212 to form a bright field light source 212 and a dark field light source 212, and can adjust strength of the dark field. The control host 3 is electrically connected to the light source 212 and can control brightness of the light source 212.

The microscope assembly 22 includes an image grabber 225, a position adjusting mechanism, and an imaging adjusting mechanism. The position adjusting mechanism is configured to adjust positions of the image grabber 225 and the imaging adjusting mechanism and includes a rotating platform 227 mounted in the middle part of the chamber body 141. A telescopic motor 226 is mounted on the rotating platform 227. The imaging adjusting mechanism is mounted at a telescopic end of the telescopic motor 226 and includes the microscope 221 and a focusing driven gear 223 and a magnification adjusting driven gear 224 both arranged on the microscope 221. The focusing driven gear 223 meshes with a focusing driving gear. The focusing driving gear is arranged on a focusing motor 222a. The magnification adjusting driven gear 224 meshes with a magnification adjusting driving gear. The magnification adjusting driving gear is arranged on a magnification adjusting motor 222b. The magnification adjusting motor 222b and the focusing motor 222a are both mounted on a mounting bracket. The magnification adjusting motor 222b and the focusing motor 222a adjust optical imaging magnification and imaging focal length of the microscope 221 respectively. The mounting bracket is arranged at the telescopic end of the telescopic motor 226. The image grabber 225 is mounted on the mounting bracket. The image grabber 225 is arranged opposite to the microscope 221. The image grabber 225, the rotating platform 227, the telescopic motor 226, the magnification adjusting motor 222b and the focusing motor 222a are all electrically connected to the control host 3. The three-dimensional coordinate positioning of the biological sample in the culture dish 131 is accomplished by means of the rotating platform 227, the telescopic motor 226, and the focusing motor 222a. The light source 212 emits light, the light passes through the biological sample contained in the culture dish 131, the hollow shaft 133, and the microscope 221 in sequence, and is ultimately captured by the image grabber 225. Thus, the image grabber captures images of the biological sample contained in the culture dish 131.

It should be finally noted that the examples above are merely intended to explain rather than limit the technical solutions of the present disclosure. Although the present disclosure has been described in detail with reference to the preferred examples, those skilled in the art should understand that modifications or equivalent substitutions may still be made to the technical solutions of the present disclosure, while such modifications or equivalent substitutions should not cause the modified technical solutions to depart from the spirit and scope of the technical solutions of the present disclosure.

What is claimed is:

1. A rotary biological culture and observation apparatus, comprising a control-display terminal and a control host, the control-display terminal being electrically connected to the control host, the rotary biological culture and observation apparatus further comprising an environmental regulation system, culture dish rotation systems and an imaging system, the culture dish rotation systems and the imaging system both being arranged inside the environmental regulation system, wherein the environmental regulation system comprises a culture chamber, the culture chamber comprises a chamber body and a door connected to the chamber body, and a plurality of culture dish rotation systems are distributed in the chamber body circumferentially.

2. The rotary biological culture and observation apparatus of claim 1, wherein a sealing gasket is arranged at a contact position between the chamber body and the door, and the culture chamber is provided with a vent.

3. The rotary biological culture and observation apparatus of claim 2, wherein a temperature regulation subsystem is arranged inside the culture chamber, the temperature regulation subsystem comprises a door temperature controller and a chamber body temperature controller, the door temperature controller is electrically connected to a first heating plate secured within the door, the chamber body temperature controller is electrically connected to a second heating plate secured within the chamber body, and the door temperature controller and the chamber body temperature controller are both electrically connected to the control host.

4. The rotary biological culture and observation apparatus of claim 3, wherein a gas concentration regulation subsystem is arranged inside the culture chamber, the gas concentration regulation subsystem comprises an intake controller, a detection assembly, and a homogenizing fan, the intake controller, the detection assembly and the homogenizing fan are all electrically connected to the control host, the homogenizing fan and the detection assembly are both mounted inside the chamber body, the detection assembly comprises an oxygen sensor and a carbon dioxide sensor, and the intake controller comprises a nitrogen gas port, an oxygen gas port, a carbon dioxide gas port, and a mixed gas outlet;
oxygen concentration regulation operations are as follows:
the nitrogen gas port is configured to be opened while the oxygen gas port is closed, and nitrogen gas is continuously fed to discharge oxygen gas from the chamber body to reduce a concentration of the oxygen gas in the chamber body; and the oxygen gas port is configured to be opened while the nitrogen gas port is closed, and the oxygen gas is continuously fed to increase the concentration of the oxygen gas in the chamber body;
carbon dioxide regulation operations are as follows:
the carbon dioxide gas port is configured to be closed while the nitrogen gas port is opened, and the nitrogen gas is continuously fed into the chamber body to reduce a concentration of carbon dioxide in the chamber body; and
the carbon dioxide gas port is configured to be opened, and the carbon dioxide is continuously fed into the chamber body to increase the concentration of the carbon dioxide in the chamber body.

5. The rotary biological culture and observation apparatus of claim 4, wherein each of the plurality of culture dish rotation systems comprises a dish holder to be connected to a culture dish, and a protective housing, the dish holder is mounted at an end of a hollow shaft, the hollow shaft is connected to the protective housing by means of two bearings, the hollow shaft between the two bearings is provided with a rotation driven gear, the rotation driven gear meshes with a rotation driving gear, the rotation driving gear is connected to a transmission shaft, the transmission shaft passes through the protective housing to be connected to a rotation drive motor, and the rotation drive motor is mounted on the protective housing;
a photoelectric switch is mounted on a rear cover plate of the protective housing, a detection plate is mounted on the hollow shaft, and the detection plate is arranged opposite to the photoelectric switch; and
the rotation drive motor and the photoelectric switch are both electrically connected to the control host.

6. The rotary biological culture and observation apparatus of claim 5, wherein the imaging system comprises light source assemblies and microscope assemblies, the light source assemblies are arranged opposite to the microscope assemblies.

7. The rotary biological culture and observation apparatus of claim 6, wherein each of the light source assemblies comprises an adjusting plate and a light source, the light source is mounted on the door and arranged opposite to the hollow shaft, the door is provided with a plurality of elongated adjusting through-holes distributed circumferentially, a limit post on the adjusting plate is arranged in a corresponding one of the plurality of elongated adjusting through-holes, and the adjusting plate is arranged between the light source and the culture dish; and
the light source is electrically connected to the control host.

8. The rotary biological culture and observation apparatus of claim 6, wherein each of the microscope assemblies comprises an image grabber, a position adjusting mechanism, and an imaging adjusting mechanism, the position adjusting mechanism comprises a rotating platform mounted in a middle part of the chamber body, a telescopic motor is mounted on the rotating platform, the imaging adjusting mechanism is mounted at a telescopic end of the telescopic motor and comprises a microscope and a focusing driven gear and a magnification adjusting driven gear both arranged on the microscope, the focusing driven gear meshes with a focusing driving gear, the focusing driving gear is arranged on a focusing motor, the magnification adjusting driven gear meshes with a magnification adjusting driving gear, the magnification adjusting driving gear is arranged on a magnification adjusting motor, the magnification adjusting motor and the focusing motor are both mounted on a mounting bracket, the mounting bracket is arranged at the telescopic end of the telescopic motor, the image grabber is mounted on the mounting bracket, and the image grabber is arranged opposite to the microscope; and
the image grabber, the rotating platform, the telescopic motor, the magnification adjusting motor and the focusing motor are all electrically connected to the control host.
